(19) United States
(12) Patent Application Publication
(10) Pub. No.: US 2023/0008522 A1
(43) Pub. Date: Jan. 12, 2023

(54) Wireless Earbud Device

(71) Applicant: EM-TECH Co., Ltd., Gyeongsangnam-do (KR)

(72) Inventors: Hyuk Won, Busan (KR); Gyu Ha Lee, Gyeonggi-do (KR)

(21) Appl. No.: 17/854,543

(22) Filed: Jun. 30, 2022

(30) Foreign Application Priority Data
Jul. 5, 2021 (KR) 10-2021-0087529

(51) Int. Cl. G01K 13/20; H04R 1/10

(52) U.S. Cl. CPC G01K 13/20; H04R 1/1016; H04R 2420/07

(57) ABSTRACT

A wireless earbud device includes at least one wireless earbud including a main body portion in which a front portion is in contact with or close to a user's concha when worn on the user's ear and an extension portion connected to a lower end of the main body portion. A sound emission hole and a temperature sensor are provided in the front portion of the main body portion of the at least one wireless earbud. A data processor mounted in an accommodation space of the main body portion is configured to estimate an eardrum temperature or a core body temperature using analysis data learned by an artificial intelligence upon receiving a first detection value from the temperature sensor.

### TECHNICAL FIELD

The present disclosure relates to a wireless earbud device for detecting a user's body temperature (especially, a core body temperature) more accurately when the user uses the wireless earbud device in contact with or close to the skin, and determining a physical condition of the user using the detected body temperature.

### BACKGROUND

Wireless sound conversion devices include audio devices such as earphones, earbuds, and headsets, and perform sound reproduction, phone calls, etc., while performing communication with an electronic device (e.g., a smartphone, a tablet, etc.) wirelessly.

The related art wireless sound conversion devices do not provide a function of maintaining health or for a treatment, other than sound reproduction or phone calls mentioned above to wearers thereof.

### SUMMARY

An aspect of the present disclosure provides a wireless earbud device for detecting a user's body temperature (especially, a core body temperature) more accurately when the user uses the wireless earbud device in contact with or close to the skin, and determining a physical condition of the user using the detected body temperature.

In an aspect, a wireless earbud device includes: at least one wireless earbud including a main body portion in which a front portion is in contact with or close to a user's concha when worn on the user's ear and an extension portion connected to a lower end of the main body portion, wherein a sound emission hole and a temperature sensor are provided in the front portion of the main body portion of the at least one wireless earbud, and a data processor is mounted in an accommodation space of the main body portion and estimates an eardrum temperature or a core body temperature using analysis data learned by an artificial intelligence upon receiving a first detection value from the temperature sensor, wherein the data processor determines a health condition according to the core body temperature using determination data, a light irradiation unit is mounted in the main body portion of the at least one wireless earbud, and the data processor performs a light irradiation function by controlling the light irradiation unit according to the determined health condition.

A proximity sensor measuring a distance to the user's skin may be mounted on the main body portion of the at least one wireless earbud, wherein the data processor may estimate the eardrum temperature or the core body temperature by correcting the first detection value in consideration of a second detection value that is the measured distance from the proximity sensor.

A motion sensor detecting a third detection value that is the user's motion amount or motion information may be mounted on the main body portion of the at least one wireless earbud, wherein the data processor may estimate the eardrum temperature or the core body temperature by correcting the first detection value in consideration of the third detection value from the motion sensor.

A photoplethysmography (PPG) sensor may be mounted on the main body portion of the at least one wireless earbud, wherein the data processor may estimate a heart rate variability from a fourth detection value that is a PPG from the PPG sensor and estimate the eardrum temperature or the core body temperature by correcting the first detection value in consideration of the estimated heart rate variability.

The data processor may estimate the eardrum temperature or the core body temperature by correcting the first detection values from a plurality of positions of each of a plurality of wireless earbuds.

The data processor may determine the user's sleep quality from continuous core body temperatures by using prediction data on a correlation between a Circadian rhythm and the core body temperature.

The data processor may read analysis data for emotion analysis and analyze the user's emotion using at least one of the core body temperature or the heart rate variability.

According to the present disclosure, when the user uses the wireless earbud device in contact with or close to the skin, light irradiation may be performed by determining a physical condition of the user (an emotional state, a sleep quality, a health condition, etc.) by estimating an eardrum temperature and/or a core body temperature more accurately from a temperature of the user's concha.

### BRIEF DESCRIPTION OF THE DRAWINGS

FIG. 1 is a perspective view of a wireless earbud device according to the present disclosure.

FIG. 2 is a configuration diagram of a body temperature measurement system including the wireless earbud device of FIG. 1.

### DETAILED DESCRIPTION

Hereinafter, various embodiments of the present disclosure will be described with reference to the accompanying drawings. Accordingly, those of ordinary skill in the art will recognize that modification, equivalent, and/or alternative on the various embodiments described herein may be variously made without departing from the scope and spirit of the present disclosure. With regard to description of drawings, similar components may be marked by similar reference numerals.

In the disclosure disclosed herein, the expressions “have”, “may have”, “include” and “comprise”, or “may include” and “may comprise” used herein indicate existence of corresponding features (for example, elements such as numeric values, functions, operations, or components) but do not exclude presence of additional features.

In the disclosure disclosed herein, the expressions “A or B”, “at least one of A or/and B”, or “one or more of A or/and B”, and the like used herein may include any and all combinations of one or more of the associated listed items. For example, the term “A or B”, “at least one of A and B”, or “at least one of A or B” may refer to all of the case (1) where at least one A is included, the case (2) where at least one B is included, or the case (3) where both of at least one A and at least one B are included.

The terms, such as “first”, “second”, and the like used herein may refer to various elements of various embodiments of the present disclosure, but do not limit the elements. For example, such terms are used only to distinguish an element from another element and do not limit the order and/or priority of the elements. For example, a first user device and a second user device may represent different user devices irrespective of sequence or importance. For example, without departing the scope of the present disclosure, a first element may be referred to as a second element, and similarly, a second element may be referred to as a first element.

It will be understood that when an element (for example, a first element) is referred to as being “(operatively or communicatively) coupled with/to” or “connected to” another element (for example, a second element), it may be directly coupled with/to or connected to the other element or an intervening element (for example, a third element) may be present. In contrast, when an element (for example, a first element) is referred to as being “directly coupled with/to” or “directly connected to” another element (for example, a second element), it should be understood that there are no intervening element (for example, a third element).

According to the situation, the expression “configured to” used herein may be used as, for example, the expression “suitable for”, “having the capacity to”, “designed to”, “adapted to”, “made to”, or “capable of”. The term “configured to (or set to)” must not mean only “specifically designed to” in hardware. Instead, the expression “a device configured to” may mean that the device is “capable of” operating together with another device or other components. For example, a “processor configured to (or set to) perform A, B, and C” may mean a dedicated processor (for example, an embedded processor) for performing a corresponding operation or a generic-purpose processor (for example, a central processing unit (CPU) or an application processor) which may perform corresponding operations by executing one or more software programs which are stored in a memory device.

Terms used in this specification are used to describe specified embodiments of the present disclosure and are not intended to limit the scope of the present disclosure. The terms of a singular form may include plural forms unless otherwise specified. Unless otherwise defined herein, all the terms used herein, which include technical or scientific terms, may have the same meaning that is generally understood by a person skilled in the art. It will be further understood that terms, which are defined in a dictionary and commonly used, should also be interpreted as is customary in the relevant related art and not in an idealized or overly formal detect unless expressly so defined herein in various embodiments of the present disclosure. In some cases, even if terms are terms which are defined in the specification, they may not be interpreted to exclude embodiments of the present disclosure.

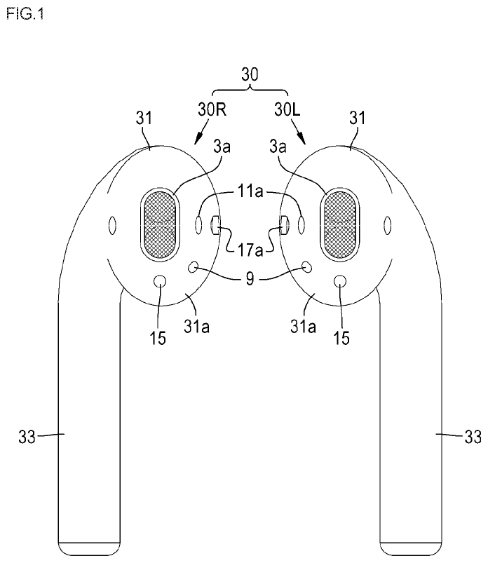

FIG. 1 is a perspective view of a wireless earbud device 30 according to the present disclosure.

The wireless earbud device 30 may include a pair of wireless earbuds 30L and 30R or a single wireless earbud. In the present embodiment, each of the wireless earbuds 30L and 30R has the same or symmetrical outer shape and is collectively referred to as a wireless earbud device 30.

The wireless earbud device 30 includes a main body portion 31 in which a front portion is in contact with or close to a concha of a wearer when worn by the wearer or a user and a rear portion is exposed to the outside and an extension portion 33 connected to a lower end of the main body portion 31 to accommodate a battery or the like.

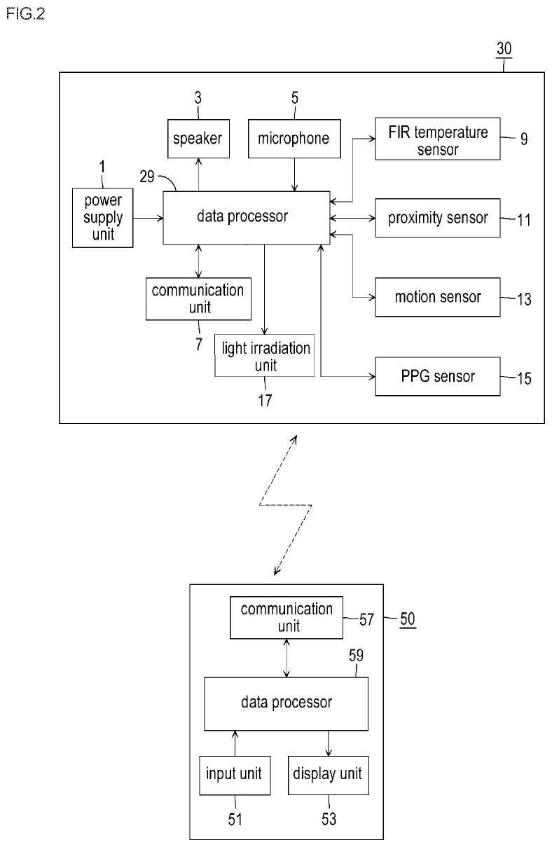

The main body portion 31 may have an accommodation space therein, and a control configuration (described in FIG. 2) of the wireless earbud device 30 is inserted into and mounted in the accommodation space. The main body portion 31 is formed to have a size enough to be inserted into the ear to be in contact with or close to the concha.

The front portion 31a of the main body portion 31 may have a sound emission hole 3a for emitting sound, a detection hole 11a formed to measure an interval between the main body portion 31 or the accommodation space of the wireless earbud device 30 and the user's skin, and through holes such as an optical through-hole for light irradiation. In addition, a far-infrared (FIR) temperature sensor 9, a photoplethysmography (PPG) sensor 15, and the like are mounted in the front portion 31a of the main body portion 31 so as to be in contact with or close to the user's skin.

The FIR temperature sensor 9 may be in contact with or close to the concha of the human body and may be located in contact with or close to other parts (e.g., forehead, etc.). Most of the ears of the human body is in contact with the outside air and are significantly affected by an external temperature to have a significant temperature difference from a core body temperature, but the concha may be in less contact with the outside air to have a temperature close to the core body temperature. The FIR temperature sensor 9 of the main body portion 31 is preferably in contact with or close to the concha to utilize the temperature characteristic of the concha.

FIG. 2 is a configuration diagram of a body temperature measurement system including the wireless earbud device 30 of FIG. 1.

The body temperature measurement system is configured to include the wireless earbud device 30 in contact with or close to the user's skin to measure a detection temperature. Each of the wireless earbud devices 30L and 30R may be configured to have the same control configuration as that of the wireless earbud device 30 of FIG. 2.

The wireless earbud device 30 includes a power supply unit 1 having a battery accommodated in the extension portion 33 and supplying power for use, a speaker 3 mounted in the accommodation space of the main body portion 31, receiving an acoustic signal from the data processor 29, and emitting a sound through the sound emission hole, a microphone 5 mounted in the accommodation space of the main body portion 31 to acquire sound and applying sound to the data processor 29, a communication unit 7 mounted in the accommodation space of the main body portion 31 and performing wireless communication with an electronic communication device 50 or other wireless earbuds, etc., an FIR temperature sensor 9 detecting a temperature and applying a detection temperature (hereinafter, referred to as a “first detection value”) to the data processor 29, a proximity sensor 11 mounted in the accommodation space of the main body portion 31, measuring a distance to the skin, and applying the measured distance (hereinafter, referred to as a “second detection value”) to the data processor 29, a motion sensor 13 (e.g., an inertial measurement unit (IMU) sensor) detecting a detection value (motion detection value) (hereinafter, referred to as a “third detection value”) for a change in an inertia and applying the detection value to the data processor 29, a photoplethysmography (PPG) sensor 15 detecting a detection value (hereinafter, referred to as a “fourth detection value”) for a PPG (or an optical blood flow rate) and applying the detection value to the data processor 29, a light irradiation unit 17 irradiating light (a near-infrared ray, etc.) through an optical through hole according to an optical control signal from the data processor 29, and the data processor 29 mounted in the accommodation space of the main body portion 31, performing a function such as sound emission, phone calls, etc. by controlling the components described above, and estimating a core body temperature of the user more precisely by correcting the first detection value. However, the power supply unit 1, the speaker 3, the microphone 5, the communication unit 7, the FIR temperature sensor 9, the proximity sensor 11, the motion sensor 13, the PPG sensor 15, and the like correspond to technologies easily recognized by those skilled in the art to which the present disclosure pertains, and thus, descriptions thereof are omitted.

The light irradiation unit 17 is controlled by the data processor 29 and includes a plurality of optical elements irradiating light in visible and near infrared bands in the range of 650 to 1,300 nm. The light irradiation unit 17 may include, for example, light emitting diode (LED) elements.

The data processor 29 is configured as an electrical or electronic circuit including a processor (e.g., a CPU, a microcontroller unit (MCU), a microprocessor, etc.) for estimating an eardrum temperature or a core body temperature more precisely by correcting the first detection value and a storage space (e.g., a memory, etc.) storing the first to fourth detection values, correction data for correcting the first detection value for estimating the core body temperature, prediction data for predicting a sleep quality using the core body temperature and a circadian rhythm, analysis data for analyzing the user's emotion using the core body temperature and a heart rate variability (HRV), light irradiation data for performing a light irradiation function for the user by determining a health condition of the user from the core body temperature, and the like.

The correction data includes at least one of first data for estimating or predicting the eardrum temperature by correcting the first detection value, second data for estimating or predicting the eardrum temperature by correcting the first detection value in consideration of the second detection value, third data for estimating or predicting the eardrum temperature by correcting the first detection value in consideration of the third detection value, fourth data for estimating or predicting the eardrum temperature by correcting the first detection value in consideration of the fourth detection value, fifth data for estimating or predicting the core body temperature from the eardrum temperature, and sixth data for estimating or predicting the core body temperature from first detection values of a plurality of positions (e.g., forehead, palm, etc.) of the human body. However, the fifth data may be essentially provided.

The first data is configured to include a formula or a data table machine-learned by artificial intelligence to compare the first detection values from the wireless earbud devices 30L and 30R to obtain an average or estimate a common detection value by applying a weight value and correct the common detection value to estimate or predict the eardrum temperature.

In addition, the second data is to solve a problem that, when the FIR temperature sensor 9 is not in contact with but spaced apart from the skin, the first detection value is affected by an external air temperature and the accuracy of the first detection value is lowered. That is, the second data is configured to include a formula or a data table machine-learned by artificial intelligence to estimate or predict the eardrum temperature by correcting the first detection value in consideration of the second detection value that is a measurement distance.

In addition, since the first data may be affected depending on the user's motion amount or degree of motion, the third data may be configured to include a formula and a data table machine-learned by artificial intelligence to estimate or predict the eardrum temperature by correcting the first detection value in consideration of the third detection value that is a detection value for a change in inertia reflecting the user's motion amount or degree of motion.

In addition, the fourth data is to consider the core body temperature affected by the influence or change of an autonomic nervous system (ANS), a peripheral nervous system (PNS), and a somatic nervous system (SNS), and the fourth data is configured to include a formula or a data table machine-learned by artificial intelligence to estimate or predict the eardrum temperature by estimating the HRV from the fourth detection value, analyzing the ANS, the PNS, and the SNS information through the estimated HRV, and correcting the first detection value in consideration of the analyzed ANS, PNS, and SNS information.

In addition, the fifth data is configured to include a formula or data table estimated by machine-learning by artificial intelligence to estimate the core body temperature from the eardrum temperature, or include a known formula.

In addition, the sixth data is configured to include a formula or data table estimated by machine-learning by artificial intelligence to estimate or predict the core body temperature from the first detection values of a plurality of positions (e.g., left and right conchas, forehead, palm, etc.) of the human body.

Next, the prediction data is configured to include a formula or a data table learned by artificial intelligence to predict the user's sleep quality from the core body temperature estimated by the correction data described above. The prediction data includes a circadian rhythm (sleep-wake circadian cycle). The circadian rhythm corresponds to a phenomenon in which a biochemical, physiological, or behavioral flow appear in a cycle of almost 24 hours not only in humans but also in living things on the Earth, including plants, animals, fungi, and even bacteria. It is known that humans have a cycle of approximately 23.5 to 24.65 hours. The core body temperature changes within the circadian rhythm, and the user's sleep quality (sleep time and quality) may also be predicted by using the change, the degree of change, or a change pattern of the core body temperature. Accordingly, the prediction data includes a formula or a data table estimated by regression-learning a correlation between continuous core body temperature information and sleep quality by artificial intelligence. The continuous core body temperature information includes at least one of core body temperatures estimated while the user sleeps and core body temperatures estimated during the user's daily routine. The FIR temperature sensor 9 applies the first detection value to the data processor 29 in real time or at regular time intervals, and the data processor 29 reads the aforementioned correction data and estimates the core body temperature corresponding to the first detection value in real time or at regular time intervals to generate continuous core body temperature information including the estimated core body temperatures.

Next, the analysis data is intended to take advantage of the fact that the user's emotional state may be analyzed from each of the core body temperature and the HRV, and the analysis data is configured to include data (e.g., an algorithm, etc.) for analyzing an emotional state from each of the core body temperature and the HRV or analyzing the emotional state by considering or reflecting both the core body temperature and the HRV.

Next, the light irradiation data is configured to include determination data for determining the user's health condition from the core body temperature and light irradiation characteristic data for performing a light irradiation function on the user to improve the determined health condition.

The health condition of the user is known to correspond to the core body temperature as shown in Table 1 below.

TABLE 1

| Core body | |
|---|---|
| temperature (° C.) | health condition |
| over 40 | Death from heatstroke |
| 37.5 to less than 40 | Hyperpyrexia, motion, and moderate fever |
| 36 to less than 37.5 | Normal range in steady state |
| 30 to less than 36 | Hypothermia |
| Less than 30 | Loss of consciousness, death due to ventricular |
| | fibrillation |

Using the data shown in Table 1, a health condition according to the core body temperature may be determined or checked. However, the range of the core body temperature is an example and may be variable. As shown in Table 1, the determination data includes data on a correspondence relationship between core body temperature and health condition.

The light irradiation characteristic data includes a wavelength of light, frequency, light irradiation time, light intensity, and the like for improving or treating the determined health condition.

The data processor 29 performs a first process of estimating or predicting more accurate core body temperature, a second process of determining a sleep quality using the core body temperature in the first process, a third process of performing an emotional analysis using the core body temperature in the first process, and a fourth process of determining a health condition using the core body temperature in the first process and performing light irradiation corresponding thereto, which will be described in detail below.

A body temperature measurement system is configured to include the wireless earbud device 30 and an electronic communication device 50 performing wireless communication.

The electronic communication device 50 is, for example, an information communication device such as a smartphone or a tablet PC, and is configured to include an input unit 51 obtaining an input (e.g., estimating a core body temperature, determining a sleep quality, analyzing emotion, performing light irradiation, etc.) from the user and applying the obtained input to a data processor 59, a display unit 53 displaying the core body temperature, the sleep quality, the analyzed emotion, the health condition, etc., a communication unit 57 performing wireless communication with the wireless earbud device 30, and the data processor 59 performing each of the first to fourth processes by controlling the aforementioned components. However, a power supply unit (not shown), the input unit 51, the display unit 53 and the like correspond to technologies easily recognized by those skilled in the art to which the present disclosure pertains, and thus, detailed descriptions thereof are omitted.

The data processor 59 is configured as an electrical or electronic circuit including a processor (e.g., a CPU, an MCU, a microprocessor, etc.) performing unique functions (e.g., phone call, video playback, etc.) and estimation of a core body temperature, analysis of a sleep quality related to the core body temperature, emotion analysis, determination of a health condition, a light irradiation function, etc. and a storage space (e.g., a memory, etc.) storing the first to fourth detection values, correction data for correcting the first detection value for estimating the core body temperature, prediction data for predicting a sleep quality using the core body temperature and a circadian rhythm, analysis data for analyzing the user's emotion using the core body temperature and a heart rate variability (HRV), light irradiation data for performing a light irradiation function for the user by determining a health condition of the user from the core body temperature, and the like described above.

The data processor 29 may transmit at least one of the first to fourth detection values, the predicted sleep quality, the analyzed emotion, and the determined health condition to the electronic communication device 50 through the communication unit 7, and the data processor 59 may receive the at least one of the first to fourth detection values, the predicted sleep quality, the analyzed emotion, and the determined health condition through the communication unit 57 and store the same.

The first process of estimating or predicting more accurate core body temperature, the second process of determining a sleep quality using the core body temperature in the first process, the third process of performing an emotional analysis using the core body temperature in the first process, and the fourth process of determining a health condition using the core body temperature in the first process and performing light irradiation corresponding thereto performed in the present disclosure may be performed in the data processor 29 of the wireless earbud 30R, the data processor 29 of the wireless earbud 30L, or the data processor 59, and the first to fourth processes performed by the data processor 29 is described sequentially as an example.

First, in the first process, the data processor 29 reads the correction data and corrects the first detection value according to the first data to estimate or predict an eardrum temperature. Subsequently, the data processor 29 estimates or predicts the core body temperature by processing the estimated or predicted eardrum temperature using the fifth data.

Also, as another embodiment of the first process, the data processor 29 reads the correction data and corrects the first detection value according to the second data to estimate or predict the eardrum temperature. Subsequently, the data processor 29 estimates or predicts the core body temperature by processing the estimated or predicted eardrum temperature using the fifth data.

Also, as another embodiment of the first process, the data processor 29 reads the correction data and corrects the first detection value according to the third data to estimate or predict the eardrum temperature. Subsequently, the data processor 29 estimates or predicts the core body temperature by processing the estimated or predicted eardrum temperature using the fifth data.

Also, as another embodiment of the first process, the data processor 29 reads the correction data and corrects the first detection value according to the fourth data to estimate or predict the eardrum temperature. Subsequently, the data processor 29 estimates or predicts the core body temperature by processing the estimated or predicted eardrum temperature using the fifth data.

Also, as another embodiment of the first process, the data processor 29 reads the correction data and corrects a plurality of first detection values according to the sixth data to estimate or predict the eardrum temperature or the core body temperature. In this case, the data processor 29 may provide the positions in which the first detection values are to be measured by voice through the speaker 3, so that each of the plurality of wireless earbuds 30R and 30L is in contact with different positions of the human body.

In the first process described above, the data processor 29 may store and use correction data directly estimated or predicted as a core body temperature without estimating or predicting the eardrum temperature from the first detection value. However, even in this case, a difference between the first detection value and the eardrum temperature, a separation interval during a non-contact with the skin, the amount of motion or the degree of motion, the HRV, a temperature difference between the plurality of detection positions, etc are also considered.

Next, in the second process, the data processor 29 reads the prediction data, analyzes the continuous core body temperature information according to the read prediction data, determines the user's sleep quality, and stores the determined sleep quality. Also, the data processor 29 may transmit the sleep quality to the electronic communication device 50 through the communication unit 7.

Next, in the third process, the data processor 29 estimates the HRV from the fourth detection value, reads the analysis data, analyzes the user's emotion using at least one of core body temperature or the HRV, and stores the analyzed emotion information. Also, the data processor 29 may transmit the analyzed emotion information to the electronic communication device 50 through the communication unit 7.

Next, in the fourth process, the data processor 29 reads the light irradiation data, determines the health condition according to the core body temperature, determines the light irradiation characteristics according to the determined health condition, and performs a light irradiation function by controlling the light irradiation unit 17 according to the determined light irradiation characteristics. The data processor 29 transmits the determined health condition to the electronic communication device 50 through the communication unit 7.

The data processor 29 repeatedly performs the first to fourth processes to continuously estimate the user's core body temperature, continuously track changes in the user's condition (the sleep quality, the health condition, the emotional state, etc.), and, in particular, performs a bio-feedback function by repeatedly performing the light irradiation function in the fourth process according to the user's condition or a change in the user's condition.

The data processor 59 obtains inputs respectively corresponding to the first to fourth processes from the user through the input unit 51 and transmits an execution command for each of the first to fourth processes to the wireless earbud device 30 through the communication unit 57. The data processor 29 of the wireless earbud device 30 receives an execution command through the communication unit 7, performs each of the first to fourth processes, and transmits results of the execution, such as the core body temperature, the health condition, the sleep quality, the emotion, etc. to the electronic communication device 50 through the communication unit 7.

According to various embodiments, at least a part of the device (e.g., modules or functions thereof) or the method (e.g., operations) may be implemented, for example, as instructions stored in a non-transitory computer-readable storage medium in a programming module form. When the instructions are executed by a processor, the processor may execute a function corresponding to the instructions. The computer-readable storage medium may be, for example, the memory.

The non-transitory computer-readable recording medium may include magnetic media such as a hard disk, a floppy disk, and a magnetic tape, optical media such as a compact disc read only memory (CD-ROM) and a digital versatile disc (DVD), magneto-optical media such as a floptical disk, and hardware devices (e.g., read only memory (ROM), random access memory (RAM), or flash memory). In addition, program instructions may include high class language codes, which may be executed in a computer by using an interpreter, as well as machine codes made by a compiler. The hardware devices described above may be configured to operate as one or more software modules to perform the operations of various embodiments, and vice versa.

A module or programming module according to various embodiments may include or exclude at least one of the above-discussed components or further include any other component. The operations performed by the module, programming module, or any other component according to various embodiments may be executed sequentially, in parallel, repeatedly, or by a heuristic method. Additionally, some operations may be executed in different orders or omitted, or any other operation may be added.

While the present disclosure has been shown and described in connection with the embodiments, it will be apparent to those skilled in the art that modifications and variations may be made without departing from the spirit and scope of the invention as defined by the appended claims.

What is claimed is:

1. A wireless earbud device, comprising:
at least one wireless earbud including a main body portion in which a front portion is in contact with or close to a user's concha when worn on the user's ear, and an extension portion connected to a lower end of the main body portion, wherein a sound emission hole and a temperature sensor are provided in the front portion of the main body portion of the at least one wireless earbud;
a data processor mounted in an accommodation space of the main body portion and configured to estimate an eardrum temperature or a core body temperature using analysis data learned by an artificial intelligence upon receiving a first detection value from the temperature sensor, wherein the data processor is configured to determine a health condition according to the core body temperature using determination data; and
a light irradiation unit mounted in the main body portion of the at least one wireless earbud,
wherein the data processor is configured to perform a light irradiation function by controlling the light irradiation unit according to the determined health condition.

2. The wireless earbud device of claim 1, further comprising:
a proximity sensor mounted on the main body portion of the at least one wireless earbud and configured to measure a distance to the user's skin,
wherein the data processor is configured to estimate the eardrum temperature or the core body temperature by correcting the first detection value in consideration of a second detection value that is the measured distance from the proximity sensor.

3. The wireless earbud device of claim 1, further comprising:
a motion sensor mounted on the main body portion of the at least one wireless earbud and configured to detect a third detection value that is the user's motion amount or motion information,
wherein the data processor is configured to estimate the eardrum temperature or the core body temperature by correcting the first detection value in consideration of the third detection value from the motion sensor.

4. The wireless earbud device of claim 1, further comprising:
a photoplethysmography (PPG) sensor mounted on the main body portion of the at least one wireless earbud,
wherein the data processor is configured to estimate a heart rate variability from a fourth detection value that is a PPG from the PPG sensor and estimate the eardrum temperature or the core body temperature by correcting the first detection value in consideration of the estimated heart rate variability.

5. The wireless earbud device of claim 4, wherein the data processor is configured to read analysis data for an emotion analysis and analyze the user's emotion using at least one of the core body temperature or the heart rate variability.

6. The wireless earbud device of claim 1, wherein the data processor is configured to estimate the eardrum temperature or the core body temperature by correcting the first detection values from a plurality of positions of each of a plurality of wireless earbuds.

7. The wireless earbud device of claim 1, wherein the data processor is configured to determine the user's sleep quality from continuous core body temperatures by using prediction data on a correlation between a circadian rhythm and the core body temperature.
